(19) United States
(12) Patent Application Publication
(10) Pub. No.: US 2011/0087233 A1
(43) Pub. Date: Apr. 14, 2011

(54) METHOD FOR GUIDING SYMMETRIC IMPLANTATION OF BONE SCREWS

(75) Inventors: Ming-Chang Wu, Guishan Shiang (TW); Hong-Yu Zhu, Guishan Shiang (TW); Hung-Sheng Tien, Guishan Shiang (TW); Kuo-Tung Kao, Guishan Shiang (TW); Chi-Bin Wu, Guishan Shiang (TW)

(73) Assignee: AccuMIS Inc., Zhonghe City (TW)

(21) Appl. No.: 12/652,755

(22) Filed: Jan. 6, 2010

(30) Foreign Application Priority Data
Oct. 9, 2009 (TW) 098134214

(51) Int. Cl. A61B 17/58

(52) U.S. Cl. 606/104

(57) ABSTRACT

A method for guiding symmetric implantation of bone screws is provided. The method includes the steps of: providing a spinal image of a spine, wherein the spine has a first bone screw implanted in one side of a spinous process of the spine; obtaining a first implanting information of the first bone screw; setting a reference line in the spinal image; generating a second implanting information by mirroring the first implanting information with respect to the reference line; and guiding a second bone screw to be implanted in the other side of the spinous process according to the second implanting information. The method generates a second implanting information that is based on and symmetric to the first implanting information, thus allowing the bone screws to be precisely implanted in both sides of the spinous process and aligned with each other in the same transverse section of the spinous process.

### BACKGROUND OF THE INVENTION

1. Technical Field

The present invention relates to methods for guiding symmetric implantation of bone screws and, more particularly, to a method for guiding symmetric implantation of bone screws into two sides of a spinous process and for use with a surgical guidance system.

2. Description of Related Art

Bone fractures, herniation of intervertebral discs (HIVD), or spondylolisthesis may occur in the human spine because of trauma or lumbar spine degeneration, and as a consequence, pain will develop from the compression of spinal nerves in the vertebral canal. The best solution to the problem is to implant a fixer in the spine. In this regard, the most common surgical technique is the pedicle screw placement (also known as posterior pedicle screw fixation).

Please refer to FIG. 1 for a perspective view of conventional pedicle screw placement, and to FIG. 2 for a front side perspective view of four bone screws and two connecting rods shown in FIG. 1.

Referring to FIG. 1, a vertical spine 10, is depicted to be horizontal (assuming that the human body is in a recumbent position). As shown in the drawing, pedicle screw implantation entails inserting bone screws 20 into pedicles of the spine 10 until the bone screws 20 are implanted in a vertebral body of the spine 10 and then coupling together each two bone screws 20 that are aligned along the spine 10 with connecting rods 21, so as to fix an interbody fusion cage 30 in position to the spine 10 effectively. In practice, precise implantation of each two bone screws 20 that flank a spinous process 11 of the spine 10 is the key factor in the success of pedicle screw placement.

Referring to FIG. 2, when implanted at the same angle and to the same depth, each two bone screws 20 that flank the spinous process 11 are aligned in the same transverse section of the spinous process 11 (indicated by the implantation range A shown in FIG. 2, wherein each two bone screws 20 that flank the spinous process 11 substantially overlap). Under such conditions, when each two bone screws 20 that are aligned along the spine 10 are subsequently coupled together by the connecting rod 21, the forces exerted on the two sides of the spinous process 11 are equal, meaning that the pedicle screw placement is performed successfully.

Success of pedicle screw placement depends on precision of implantation and the surgeon's clinical experience. Spinal cord injuries can occur from a slight error in the implantation path of a bone screw. Conventionally, the surgeon diagnoses spinal conditions and plans implantation paths with the assistance of two-dimensional computed tomography (CT). However, the absolute and relative positions of the vertebrae during operation are likely to differ from those shown by pre-operative CT scanning, and thus the implantation paths during operation may deviate from those previously conceived by the surgeon. The problems can only be solved by the surgeon's professional judgment and ample experience. Nonetheless, each of the aforesaid factors is a reminder of the uncertainty and difficulty of pedicle screw placement.

### BRIEF SUMMARY OF THE INVENTION

The present invention provides a method for guiding symmetric implantation of bone screws, embodies the notion of computer-aided surgery, and applies to a surgical guidance system for use with pedicle screw placement. The method involves generating a second implanting information that is based on and symmetric to a first implanting information of a first bone screw, so that bone screws implanted in two sides of a spinous process are precisely aligned with each other in the same transverse section of the spinous process. Thus, the forces exerted on the two sides of the spinous process are equal, the bone screws are prevented from loosening, dislocating, or breaking, and as a consequence, the success rate of pedicle screw placement is significantly improved.

To achieve the above and other objectives, the present invention provides a method that is designed to guide symmetric implantation of bone screws and is applicable to a surgical guidance system. The method includes the steps of: providing a spine image of a spine, wherein the spine has a first bone screw implanted in one side of a spinous process of the spine; obtaining a first implanting information of the first bone screw; setting a reference line in the spine image; generating a second implanting information by mirroring the first implanting information with respect to the reference line; and guiding a second bone screw to be implanted in the other side of the spinous process according to the second implanting information.

Implementation of the present invention at least involves the following progressive aims:

1. The method disclosed herein embodies the notion of computer-aided surgery and uses a spinal image as a reference interface so as to generate a second implanting information that is based on and symmetric to a first implanting information of a first bone screw and is subsequently used to guide the implantation of a second bone screw.

2. The implanting information of a bone screw to be implanted on the other side of a spinous process is generated by a mirroring process so that bone screws flanking the spinous process can be implanted in the same transverse section of the spinous process. As a result, the forces exerted on the two sides of the spinous process are equal so as to prevent the bone screws from loosening, dislocating, or breaking, and therefore the success rate of pedicle screw placement is increased.

### BRIEF DESCRIPTION OF THE DRAWINGS

The features and advantages of the present invention are described in detail hereunder to enable a person skilled in the art to understand and implement the technical content of the present invention and readily comprehend the objectives and advantages of the present invention by reviewing the disclosure of the specification, the appended claims, and the accompanying drawings, in which:

FIG. 1 is a perspective view of conventional pedicle screw placement;

FIG. 2 is a front side perspective view of four bone screws and two connecting rods shown in FIG. 1;

FIG. 3 is a flowchart of a method for guiding symmetric implantation of bone screws according to the present invention; and

FIG. 4A through FIG. 4D are schematic views for illustrating the steps shown in FIG. 3.

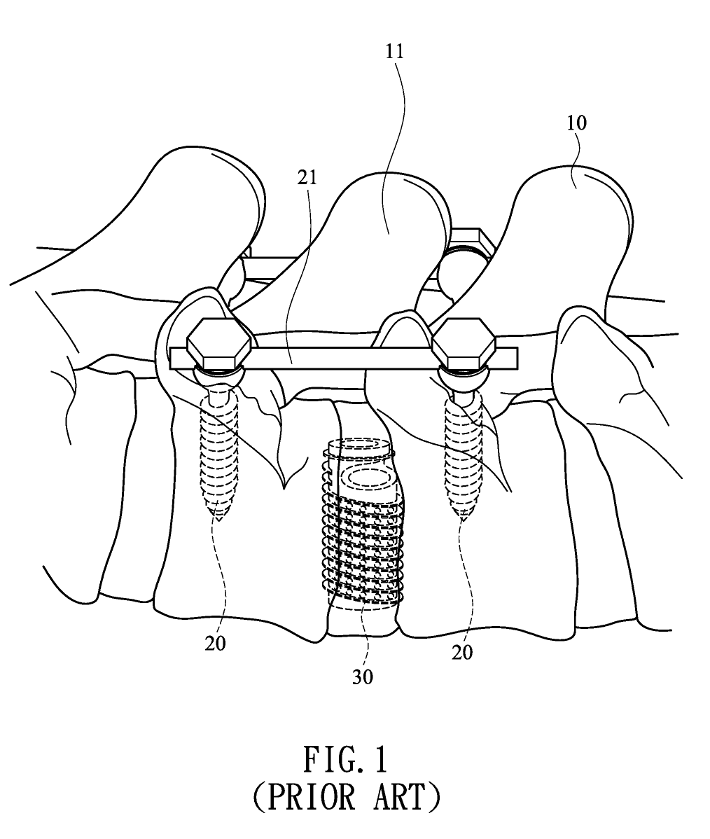

### DETAILED DESCRIPTION OF THE INVENTION

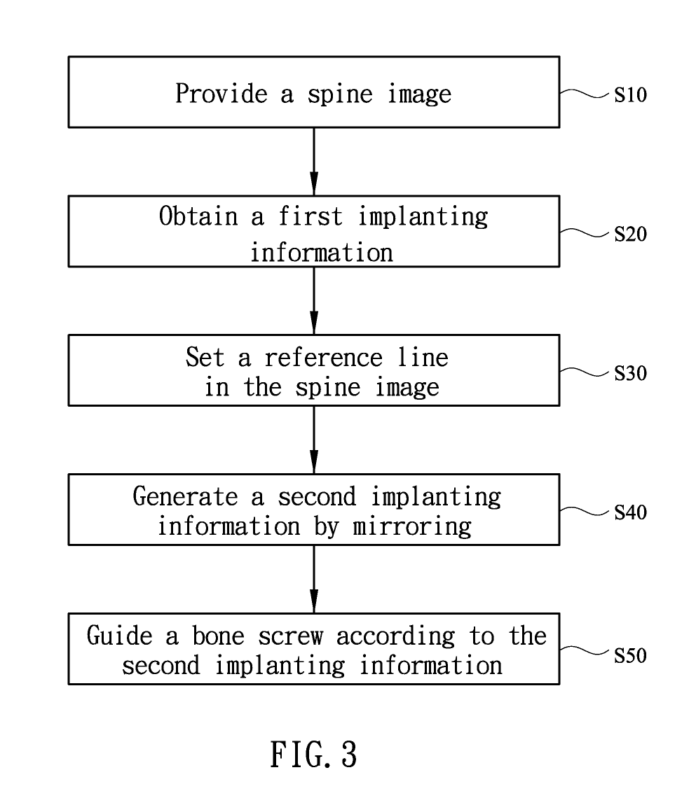

Please refer to FIG. 3 for a flowchart of a method for guiding symmetric implantation of bone screws according to an embodiment of the present invention. The method is applicable to a surgical guidance system for use with pedicle screw placements and includes the steps of: providing a spinal image (S10); obtaining a first implanting information (S20); setting a reference line in the spinal image (S30); generating a second implanting information by mirroring (S40); and guiding a bone screw according to the second implanting information (S50).

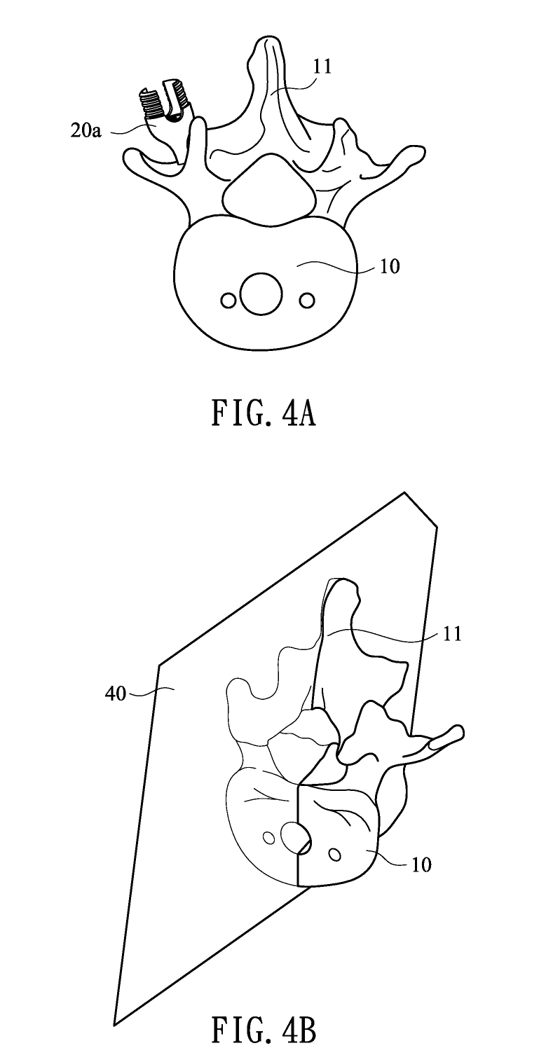

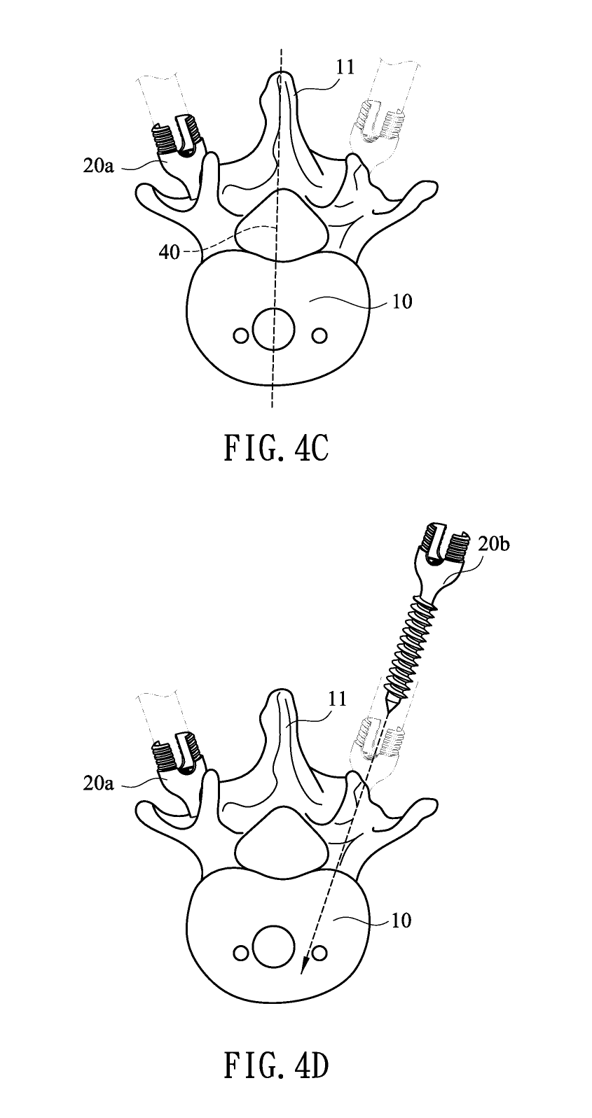

Please refer to FIG. 4A through FIG. 4D for the flowchart of the steps illustrated corresponding to FIG. 3. More specifically, FIG. 4A corresponds to steps S10 and S20 shown in FIG. 3 while FIGS. 4B, 4C, and 4D correspond to steps S30, S40, and S50, respectively.

At the step of providing a spinal image (S10), as shown in FIG. 4A, an image of a spine 10 is provided. The spine 10 has a first bone screw 20a implanted in one side of a spinous process 11 of the spine 10. Hence, the spinal image includes images of the spine 10 and the first bone screw 20a.

The step of obtaining a first implanting information (S20) is detailed with reference to FIG. 4A. Now that the first bone screw 20a is already implanted in the spine 10, it is feasible to use a surgical guidance system to record and analyze the spinal image so as to extract an implantation angle and an implantation depth of the first bone screw 20a as a first implanting information. The first implanting information is stored in a database of the surgical guidance system for future retrieval.

At the step of setting a reference line in the spinal image (S30), as shown in FIG. 4B, a spinous process section 40 of the spinous process 11 is taken along the spine 10 from the spinal image and set as a reference line. When shown in a perspective view, the reference line defines a sagittal plane that bilaterally divides the spinous process 11 into two symmetric halves.

The step of generating a second implanting information by mirroring (S40) is carried out in the following manner. As shown in FIG. 4C, with the spinous process section 40 serving as the reference line, the first implanting information is retrieved from the database of the surgical guidance system and mirrored, with respect to the reference line, with the other side of the spinous process 11 so as to generate a second implanting information that includes a corresponding implantation angle and depth. The second implanting information is used to set out the implantation path of a second bone screw 20b in the other side of the spinous process 11.

The step of guiding a bone screw according to the second implanting information (S50) is now explained with reference to FIG. 4D. Implantation of the second bone screw 20b into the other side of the spinous process 11 is guided according to the second implanting information such that the bone screws implanted on both sides of the spinous process 11 are precisely aligned in the same transverse section of the spinous process 11. Thus, the forces exerted on the two sides of the spinous process 11 will be equal.

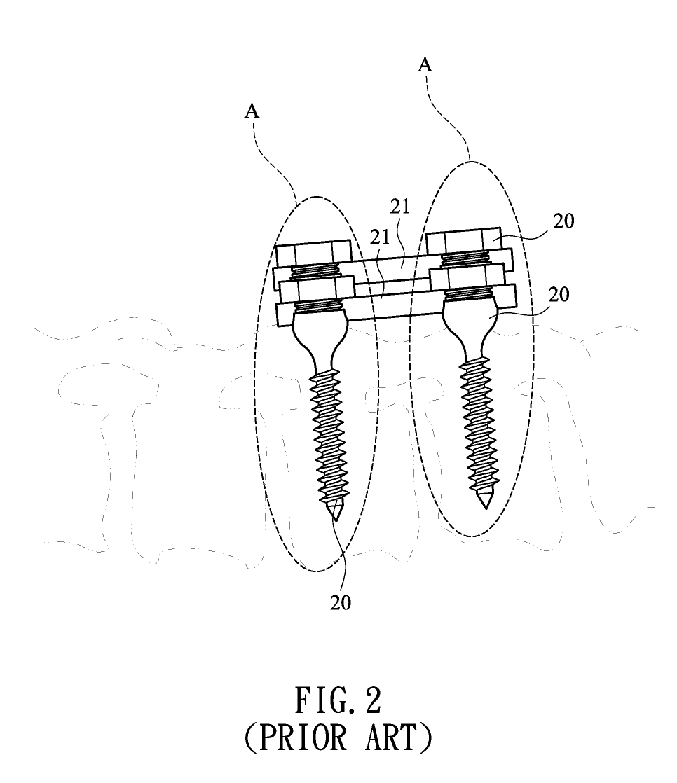

Since the second implanting information mirrors the first implanting information, the first bone screw 20a and the second bone screw 20b have corresponding implantation depths and implantation angles, i.e., the implantation depths and implantation angles of the two bone screws 20a and 20b are bilaterally symmetrical with respect to the spinous process section 40. When shown in a side perspective view of the spine 10, such as FIG. 2, the bone screws that flank the spinous process 11 substantially overlap and are aligned in the same transverse section of the spinous process 11, thus allowing equal forces to be exerted on the two sides of the spinous process 11. Consequently, the bone screws are prevented from loosening, dislocating, or breaking, and the success rate of pedicle screw placement is significantly improved.

The foregoing embodiment is illustrative of the characteristics of the present invention so as to enable a person skilled in the art to understand the disclosed subject matter and implement the present invention accordingly. The embodiment, however, is not intended to restrict the scope of the present invention. Hence, all equivalent modifications and variations of the foregoing embodiment that do not depart from the spirit and principle of the present invention should fall within the scope of the appended claims.

What is claimed is:

1. A method for guiding symmetric implantation of bone screws, the method being applicable to a surgical guidance system and comprising steps of:
providing a spinal image of a spine, wherein the spine has a first bone screw implanted in one side of a spinous process of the spine;
obtaining a first implanting information of the first bone screw;
setting a reference line in the spinal image;
generating a second implanting information by mirroring the first implanting information with respect to the reference line; and
guiding a second bone screw to be implanted in the other side of the spinous process according to the second implanting information.

2. The method of claim 1, wherein the reference line is a spinous process section taken along the spinous process in the spinal image.

3. The method of claim 1, wherein the first implanting information is obtained from a database of the surgical guidance system.

4. The method of claim 1, wherein the first implanting information comprises an implantation angle and an implantation depth.

5. The method of claim 1, wherein the second implanting information comprises an implantation angle and an implantation depth.
